(19) United States
(12) Patent Application Publication
(10) Pub. No.: US 2025/0074771 A1
(43) Pub. Date: Mar. 6, 2025

(54) NITROGEN MONOXIDE GENERATOR

(71) Applicants: Irène GABAY-GARCIA, Geneve (CH); Laura GABAY, Geneve (CH)

(72) Inventors: Irène GABAY-GARCIA, Geneve (CH); Laura GABAY, Geneve (CH)

(21) Appl. No.: 18/952,104

(22) Filed: Nov. 19, 2024

Related U.S. Application Data
(63) Continuation of application No. PCT/EP2023/063386, filed on May 17, 2023.

(30) Foreign Application Priority Data
May 19, 2022 (FR) 22/04774

(51) Int. Cl. C01B 21/20; A61K 33/00

(52) U.S. Cl. CPC C01B 21/203; A61K 33/00; C01B 2203/169

(57) ABSTRACT

The corona-effect reactor includes: a reaction chamber including an air supply port, an exhaust port, an electrode port, and a reaction cavity, the supply port, the exhaust port, and the electrode port each emerging into the reaction cavity; an air supply device configured to be fluidly connected to the air supply port by means of a guide duct and to supply air to the reactor; a high voltage electrode configured to be at least partly inside and to cooperate with the electrode port; a power supply configured to supply power to the high voltage electrode as well as the air supply device. A ratio between a cross section of the guide duct and a cross section of the reaction cavity being between ⅕ and 3/10, and for example ¼.

### CROSS-REFERENCE TO RELATED APPLICATIONS

This application is a continuation of International Application No. PCT/EP2023/063386, filed on May 17, 2023, which claims priority to and the benefit of French Patent Application No. 22/04774, filed on May 19, 2022. The disclosures of the above applications are incorporated herein by reference.

### FIELD

The present disclosure relates to a system for generating nitrogen monoxide, and more particularly to a corona-effect reactor configured to generate a gas stream which is enriched with nitrogen monoxide from an air stream.

The invention finds a preferred, and non-limiting, application in the medical and biological fields. Nitrogen monoxide is notably used as an anesthetic gas for insects, or even in medicine as an inhaled vasodilator for example.

### BACKGROUND

The statements in this section merely provide background information related to the present disclosure and may not constitute prior art.

It is known from the prior art to use a device including at least one electrode which is presented near an air stream in order to generate nitrogen monoxide; the electrode is then supplied with a high voltage current in order to transform the air stream into plasma. The devices described in the prior art generally include: an electrode whose design is based on a modified spark plug: the modification involving the removal of the ground electrode; a reaction chamber configured to receive the electrode; an air supply system configured to provide air into the reaction chamber; and a power supply system configured to power at least the electrode.

In use, it turns out that the devices for generating nitrogen monoxide as described in the prior art have certain drawbacks. The first drawback lies in the low energy efficiency as well as the degradation of the reaction chamber caused by the electric arc and the air stream in the plasma state. Indeed, the reaction inside the reaction chamber consumes a large amount of energy which is partially restored in the form of heat, which results in damage to the reaction chamber during prolonged use. Furthermore, the difficulty of using the devices as described in the prior art lies in the instability of the nitrogen monoxide concentration at the outlet of the reaction chamber.

### SUMMARY

This section provides a general summary of the disclosure and is not a comprehensive disclosure of its full scope or all of its features.

The present invention aims to remedy all or part of the drawbacks mentioned above.

The technical problem underlying the invention is in particular to provide a device for generating nitrogen monoxide which is simple and economical in structure, and whose performance is stable and durable during prolonged use.

For this purpose, the present invention concerns a corona-effect reactor, configured for the generation of nitrogen monoxide, and used in particular for the anesthesia of invertebrate animals. The reactor includes at least: a reaction chamber, which includes at least one air supply port, one exhaust port, one electrode port, and one reaction cavity, the supply port, the exhaust port, and the electrode port each emerging into the reaction cavity, the exhaust port being configured to ensure the fluid passage for an enriched stream; an air supply device, which is configured to be fluidly connected to the air supply port by means of a guide duct, and to supply air to the reaction chamber; a high voltage electrode that is configured to be at least partially within the reaction chamber and to cooperate with the electrode port; and a power supply configured to supply power to the high voltage electrode and the air supply device. The ratio between a cross section of the guide duct in the direction of the supply port and a cross section of the reaction cavity is between 0.2 and 0.3, and for example 0.25.

Advantageously, the ratio substantially close to or equal to 0.25 between the cross section of the air supply port and the cross section of the reaction cavity allows the creation of a Venturi effect as well as the expansion of an air stream coming from the air supply device when it arrives in the reaction chamber. The Venturi effect as well as the expansion of the air stream combined with the power supply of the high voltage electrode makes it possible to generate a corona effect and thus produce nitrogen monoxide from the air stream entering the reaction chamber.

Also, the specific characteristics of the invention described above make it possible to recover through the exhaust port an enriched stream which is enriched with nitrogen monoxide compared to the air stream and whose nitrogen monoxide concentration is comprised between 450 and 1000 ppm.

By high voltage electrode is meant an electrode whose operating range is comprised between 4 KV and 12 KV, and advantageously between 6 KV and 9 KV volts, and for example 7 KV, the high voltage electrode being furthermore subjected to an intensity comprised between 20 mA and 40 mA.

The corona-effect reactor may further have one or more of the following characteristics, which may be taken alone or in combination.

According to one embodiment of the present disclosure, the corona-effect reactor is also configured to carry out the generation of nitrogen monoxide by the corona effect as well as by an electric arc effect. The two phenomena are simultaneous inside the reaction chamber.

According to one embodiment of the present disclosure, the air supply device is fluidly connected to the air supply port by means of the guide duct, the guide duct being able to be cylindrical for example. Such a characteristic of the guide duct makes it possible to generate a localized Venturi effect and thus relax the air stream when it arrives in the reaction chamber.

According to one embodiment of the present disclosure, the guide duct has a passage section whose ratio between the passage section and the cross section of the reaction cavity is comprised between ⅕ and 3/10, and for example ¼.

According to one embodiment of the present disclosure, the reaction cavity is substantially cylindrical and extends along an extension axis A.

According to one embodiment of the present disclosure, the air supply port and the electrode port are substantially centered about the extension axis A.

According to one embodiment of the present disclosure, the electrode port is a tapped port, and the high voltage electrode has at least one threaded part. The tapped port being configured to complementarily cooperate with the threaded part of the high voltage electrode.

According to one embodiment of the present disclosure, the high voltage electrode is for example a modified spark plug.

According to one embodiment of the present disclosure, the modified spark plug consists of a spark plug from which the ground electrode is removed, the walls of the reaction chamber acting as ground.

According to one embodiment of the present disclosure, the air supply device is configured to supply the reaction chamber with an air stream whose flow is laminar.

The air stream first passes through the guide duct and the air supply port whose diameter is smaller than the diameter of the reaction chamber. These dimensions were found empirically after multiple tests in order to arrive at an optimum ratio between the diameter of the air supply elements and that of the reaction chamber. This ratio was defined by a diameter of the guide duct which would be ½ of that of the reaction chamber. If we now refer to the cross section by calculating its area, the ratio between the cross section of the guide duct and the cross section of the reaction chamber is close to ¼. When the continuous air passes through the conduction pipe and the reaction chamber a Venturi effect occurs with an increase in laminar air pressure in the reaction chamber.

Advantageously, the laminar flow of the air stream at the inlet of the reaction chamber allows a stable and efficient generation of nitrogen monoxide. The laminar state of the air stream obtained by the generated Venturi effect makes it possible to guarantee the homogeneity of the reaction and consequently the stability of the nitrogen monoxide concentration at the outlet of the reaction chamber.

According to one embodiment of the present disclosure, the air supply device is an electric pump, such as a membrane pump for example.

According to one embodiment of the present disclosure, the reaction chamber is made of a non-magnetic material.

According to one embodiment of the present disclosure, the reaction chamber is made of a material which is capable of electrical conduction and of creating an electric arc with the electrode.

According to one embodiment of the present disclosure, the reaction chamber is made of a material which is non-magnetic, capable of electrical conduction and which is also capable of dissipating at least in part the thermal energy generated by the corona-effect reactor during the power supply of the high voltage electrode and therefore the generation of nitrogen monoxide, the reaction chamber being made of aluminum for example.

Advantageously, the use of aluminum in order to carry out the reaction chamber makes it possible to create a corona effect but also an electric arc inside the reaction chamber. In addition, the thermal conductivity properties of aluminum represent an advantage in the temperature differences reached during the formation of the corona effect with the electric arc and the exhausting of heat in the intermittency of the power supply.

According to one embodiment of the present disclosure, the diameter of the corona radiation is 1 cm.

According to one embodiment of the present disclosure, the length of the electric arc is comprised between 0.5 and 2.5 cm.

According to one embodiment of the present disclosure, the air supply port is located opposite the exhaust port.

According to one embodiment of the present disclosure, the corona-effect reactor further includes a voltage booster configured to supply power to the high voltage electrode.

According to one embodiment of the present disclosure, the voltage booster is a switching voltage regulator. Advantageously, the switching voltage regulator has an efficiency comprised between 60% and 90%. This efficiency is to be compared with an efficiency in the range of 40% to 50% when using a linear voltage regulator. Furthermore, the footprint of the switching regulator is smaller than the footprint of the linear voltage regulator.

According to one embodiment of the present disclosure, the corona-effect reactor also includes a human-machine interface configured to simultaneously power the high voltage electrode and the air supply device when a user actuates the human-machine interface.

According to one embodiment of the present disclosure, the human-machine interface can be a pneumatic contact type switch for example.

According to one embodiment of the present disclosure, the corona-effect reactor further includes a regulating device provided downstream of the exhaust port.

According to one embodiment of the present disclosure, the regulating device is provided directly at the outlet of the exhaust port.

According to one embodiment of the present disclosure, the regulating device is a shutter which is provided downstream of the exhaust port.

According to one embodiment of the present disclosure, the regulating device is configured to occupy a plurality of positions between a closed position in which the regulating device completely shuts off the passage of at least part of the enriched stream coming from the reaction chamber, and an open position in which the regulating device does not shut off the passage of said at least part of the enriched stream coming from the reaction chamber.

According to one embodiment of the present disclosure, the regulating device is a regulating valve whose opening and/or closing is electrically piloted, such as a solenoid valve for example.

According to one embodiment of the present disclosure, the corona-effect reactor further includes a control and regulation system configured to control the production of nitrogen monoxide in the chamber by acting, as needed, on the power supply: of the high voltage electrode, of the air supply device, of the regulating device, and of the voltage booster.

According to one embodiment of the present disclosure, the corona-effect reactor further includes a selectivity device provided downstream of the exhaust port, the selectivity device being configured to act on the nitrogen monoxide concentration as well as on the products derived from nitrogen oxide which may be present in the enriched stream.

According to one embodiment of the present disclosure, the selectivity device is composed at least in part of a material capable of performing a reduction reaction of nitrogen monoxide as well as products derived from nitrogen oxide.

According to one embodiment of the present disclosure, the selectivity device is composed at least in part of soda lime for example.

Advantageously, the nitrogen monoxide concentration in the enriched stream downstream of the selectivity device can vary according to the user's needs, between 50 ppm and 400 ppm for example.

According to one embodiment of the present disclosure, the reaction chamber has an external volume whose footprint is less than 30 cm³.

According to one embodiment of the present disclosure, the reaction cavity has a general T-shaped shape.

According to one embodiment, the mass of the corona-effect reactor may be, for example, comprised between 60 and 100 grams.

Advantageously, the small footprint, the low weight, as well as the low electrical consumption required for operation make the corona-effect reactor portable, i.e. transportable by hand by the user.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

The aims, aspects and advantages of the present invention will be better understood from the description given below of a particular embodiment of the invention presented by way of non-limiting example, with reference to the accompanying drawings in which:

FIG. 1 is a schematic representation of a corona-effect reactor according to one embodiment of the present disclosure;

FIG. 2 is a representation of a high voltage electrode according to the embodiment of the present disclosure;

FIG. 3 is a schematic representation of the corona-effect reactor according to a first variant of the embodiment of the present disclosure;

FIG. 4 is a schematic representation of the corona-effect reactor according to a second variant of the embodiment of the present disclosure; and

FIG. 5 is a side perspective view of the corona-effect reactor according to the embodiment of the present disclosure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

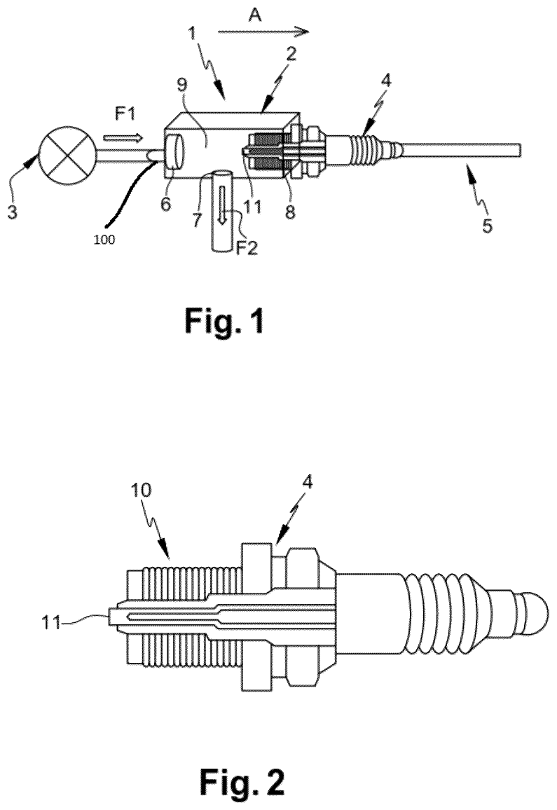

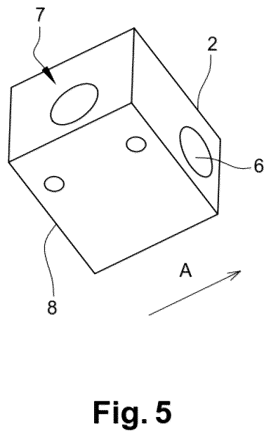

FIGS. 1, 2 and 5 represent a corona-effect reactor 1 as well as the elements constituting it configured for the generation of nitrogen monoxide according to one embodiment of the present disclosure, as well as some of the elements constituting it. The corona-effect reactor 1 includes a reaction chamber 2, an air supply device 3, a high voltage electrode 4, and a power supply 5.

The reaction chamber 2 is made of a non-magnetic material capable of electrical conduction, such as aluminum for example. The reaction chamber 2 includes an air supply port 6, an exhaust port 7, an electrode port 8, and a reaction cavity 9. The air supply port 6, the exhaust port 7 and the electrode port 8 each emerge into the reaction cavity 9. The air supply device 3 is fluidly connected to the air supply port 6 by means of a guide duct 100 which may be cylindrical for example. Such a characteristic of the guide duct 100 makes it possible to generate a localized Venturi effect and thus relax the air stream when it arrives in the reaction chamber.

The ratio between a cross section of the air supply port 6 and a cross section of the reaction cavity 9 being comprised between ⅕ and 3/10, and is for example ¼. The air supply port 6 is located opposite the electrode port 8. By cross section is meant a section which perpendicularly intersects an extension axis A. The reaction chamber 2 has an outer shape similar to a rectangular parallelepiped whose volume is less than 30 cm³; while the interior of the reaction chamber 2 forming the reaction cavity is substantially cylindrical and extends along the extension axis A.

According to the embodiment of the present disclosure as represented in the figures, the reaction cavity 9 has a general shape which can be likened to a T. The reaction cavity 9 is delimited by the reaction chamber 2, the air supply port 6, the exhaust port 7, as well as by the electrode port 8.

According to the embodiment of the present disclosure as represented in FIG. 1, the air supply device 3 is fluidly connected to the air supply port 6 and is configured to supply air to the reaction cavity 9. The air supply device 3 is configured to supply the reaction chamber 2 with an air stream F1 whose flow is laminar. Advantageously, the air supply device 3 is composed of an electric pump, such as a membrane pump for example.

According to the embodiment of the present disclosure and as shown more specifically in FIG. 2, the high voltage electrode 4 includes a threaded part 10 which is configured to complementarily cooperate with a tapped part of the electrode port 8. Also, the high voltage electrode 4 includes a discharge end 11 which is provided on the side of the threaded part 10. The discharge end 11 is configured to at least partially delimit the reaction cavity 9. By high voltage electrode 4 is meant an electrode whose operating range is comprised between 4 KV and 12 KV, and advantageously between 6 KV and 9 KV, and such as for example 7 KV, and whose operating intensity is comprised between 20 and 40 mA. Advantageously and in order to limit costs, the high voltage electrode 4 is a modified spark plug from which the ground electrode is removed.

The power supply 5 is configured to supply power to the high voltage electrode 4 as well as the air supply device 5. Advantageously, the power supply 5 of the high voltage electrode 4 makes it possible to generate a corona effect inside the reaction cavity 9. The corona effect makes it possible to transform the air stream F1 coming from the air supply port 6 into plasma, and also makes it possible to recover by the exhaust port 7 an enriched stream F2 which is enriched with nitrogen monoxide compared to the air stream F1 and whose nitrogen monoxide concentration is comprised between 450 and 1000 ppm.

Advantageously, the ratio between the cross section of the air supply port 6 and the cross section of the reaction cavity 9 allows an expansion of the air stream F1 coming from the air supply device 3. Also, the laminar flow of the air stream F1 at the inlet of the reaction chamber 2 allows a stable and efficient generation of nitrogen monoxide. The laminar state of the air stream F1 makes it possible to guarantee the homogeneity of the reaction and consequently the stability of the nitrogen monoxide concentration downstream of the exhaust port 7. Furthermore, the reaction chamber 2 which is made of a non-magnetic material capable of electrical conduction such as aluminum, makes it possible to dissipate at least part of the thermal energy generated by the corona-effect reactor 1 during the reaction.

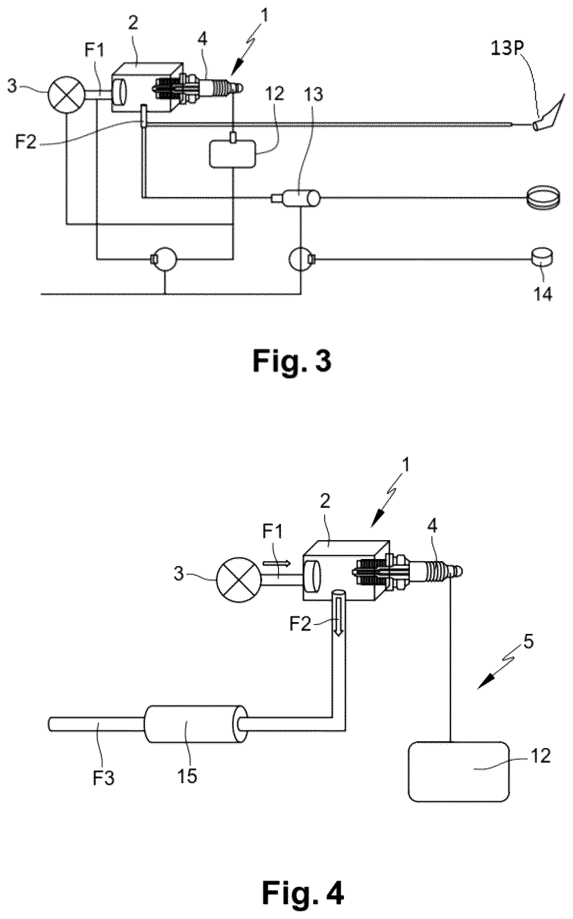

According to a first variant of the embodiment of the present disclosure which is represented in FIG. 3, the corona-effect reactor 1 further includes a voltage booster 12, a regulating device 13, and a human-machine interface 14.

The voltage booster 12 is configured to supply power to the high voltage electrode 4. The used voltage booster 12 is a switching voltage regulator. Advantageously, the switching voltage regulator has an efficiency comprised between 60% and 90%. This efficiency is to be compared with the efficiency in the range of 40% to 50% when using a linear voltage regulator. Furthermore, the footprint of the switching regulator is smaller than the footprint of the linear voltage regulator.

The regulating device 13 is provided downstream of the exhaust port 7. The regulating device 13 is configured to occupy a plurality of positions between a closed position in which the regulating device 13 completely shuts off the passage of at least part of the enriched stream F2 coming from the reaction chamber 2, and an open position in which the regulating device 13 does not shut off the passage of said at least part of the enriched stream F2 coming from the reaction chamber 2. The regulating device 13 according to the first variant of the embodiment is a regulating valve whose opening and/or closing is electrically piloted, such as a solenoid valve for example.

As more specifically visible in FIG. 3, the regulating device 13 may be a shutter, such as a gun 13P or an air blower for example, which is provided downstream of the exhaust port 7. As long as the shutter is closed, the power supply 5 and/or the voltage booster 12 do not operate. At the moment when the shutter opens, by pressing the gun for example, the system is open and the power supply 5 and/or the voltage booster 12 operate in order to produce the nitrogen monoxide which exits through the gun 13P. Conversely, releasing the trigger of the gun, and therefore closing the shutter, conditions the stopping of the production of nitrogen monoxide.

The human-machine interface 14 is configured to simultaneously power the voltage booster 12 and the air supply device 3 when a user actuates the human-machine interface 14. Also, the actuation of the human-machine interface 14 by the user initiates the transition to the open position of the regulating device 13 and/or the gun 13P. Conversely, the regulating device 13 and/or the gun 13P occupies its closed position when it is not requested by the human-machine interface 14. Advantageously, the human-machine interface may be a pneumatic contact type switch for example.

According to a second variant of the embodiment of the present disclosure represented in FIG. 4, the corona-effect reactor 1 also includes a selectivity device 15 provided downstream of the exhaust port 7. The variant of the present disclosure presented in FIG. 4 differs from the variant presented in FIG. 3 in particular in that the system is always open. The regulation of nitrogen monoxide production is obtained in particular by activating the voltage booster 12 sequentially. The selectivity device 15 is configured to act on the nitrogen monoxide concentration as well as on the nitrogen oxide derivatives present in the enriched stream F2. The selectivity device 15 is composed at least in part of a material capable of performing a reduction reaction of nitrogen monoxide and nitrogen oxide derivatives, such as soda lime for example. Advantageously, the nitrogen monoxide concentration of an outgoing flow F3 downstream of the selectivity device 15 can vary between 50 and 400 ppm, depending on the needs of the user and the dilution in the air of a ventilation circuit for example.

According to one embodiment, the mass of the corona-effect reactor may be, for example, comprised between 50 and 100 grams.

Advantageously, the small footprint, the low weight, as well as the low electrical consumption required for operation make the corona-effect reactor portable, i.e. transportable by hand by the user.

The transformation of ambient air into a gas enriched with nitrogen monoxide allows, among other things, the anesthesia of invertebrate animals such as laboratory flies, but also the use of nitrogen monoxide for therapeutic purposes for example.

Of course, the invention is in no way limited to the embodiment described and illustrated, which has been given only as an example. Modifications remain possible, in particular from the point of view of the constitution of the various elements or by substitution of technical equivalents, without departing from the scope of protection of the invention.

Unless otherwise expressly indicated herein, all numerical values indicating mechanical/thermal properties, compositional percentages, dimensions and/or tolerances, or other characteristics are to be understood as modified by the word “about” or “approximately” in describing the scope of the present disclosure. This modification is desired for various reasons including industrial practice, material, manufacturing, and assembly tolerances, and testing capability.

As used herein, the phrase at least one of A, B, and C should be construed to mean a logical (A OR B OR C), using a non-exclusive logical OR, and should not be construed to mean “at least one of A, at least one of B, and at least one of C.”

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. A corona-effect reactor configured for generation of nitrogen monoxide and used in particular for anesthesia of invertebrate animals, the corona-effect reactor comprising:
a reaction chamber, which includes at least one air supply port, one exhaust port, one electrode port, and one reaction cavity, the air supply port, the exhaust port and the electrode port each emerging into the reaction cavity, the exhaust port being configured to ensure fluid passage of an enriched stream;
an air supply device configured to be fluidly connected to the air supply port by means of a guide duct, and to supply air to the reaction chamber;
a high voltage electrode configured to be at least partly inside the reaction chamber and to cooperate with the electrode port; and
a power supply configured to supply power to the high voltage electrode as well as the air supply device,
wherein a ratio between a cross section of the guide duct in a direction of the air supply port and a cross section of the reaction cavity is between ⅕ and 3/10.

2. The corona-effect reactor according to claim 1, which is also configured to carry out the generation of nitrogen monoxide by simultaneous combination of a corona effect and an electric arc inside the reaction chamber.

3. The corona-effect reactor according to claim 1, wherein the air supply device is configured to supply the reaction chamber with an air stream whose flow is laminar.

4. The corona-effect reactor according to claim 1, wherein the reaction chamber is made of a non-magnetic material.

5. The corona-effect reactor according to claim 1, wherein the reaction chamber is made of a material which is capable of electrical conduction.

6. The corona-effect reactor according to claim 1, wherein the power supply further comprises a voltage booster.

7. The corona-effect reactor according to claim 6, wherein the voltage booster is a switching voltage regulator.

8. The corona-effect reactor according to claim 1, wherein the air supply port is located opposite the electrode port.

9. The corona-effect reactor according to claim 1, further comprising a human-machine interface configured to simultaneously power the high voltage electrode and the air supply device when a user actuates the human-machine interface.

10. The corona-effect reactor according to claim 1, further comprising a regulating device provided downstream of the exhaust port.

11. The corona-effect reactor according to claim 10, wherein the regulating device is configured to occupy a plurality of positions between a closed position in which the regulating device completely shuts off the fluid passage of at least part of the enriched stream coming from the reaction chamber, and an open position in which the regulating device does not shut off the fluid passage of said at least part of the enriched stream coming from the reaction chamber.

12. The corona-effect reactor according to claim 1, further comprising a control and regulation system configured to control production of nitrogen monoxide in the chamber by acting, as needed, on the power supply: of the high voltage electrode, of the air supply device, of the regulating device, and of a voltage booster.

13. The corona-effect reactor according to claim 1, further comprising a selectivity device provided downstream of the exhaust port, the selectivity device being configured to act on concentration of nitrogen monoxide present in the enriched stream.

14. The corona-effect reactor according to claim 1, wherein the ratio between the cross section of the guide duct in the direction of the air supply port and the cross section of the reaction cavity is ¼.
